(19) United States
(12) Patent Application Publication
(10) Pub. No.: US 2021/0023000 A1
(43) Pub. Date: Jan. 28, 2021

(54) TOPICAL COMPOSITIONS

(71) Applicant: Skin Medicinals LLC, Chicago, IL (US)

(72) Inventors: Ashvin Garlapati, Chicago, IL (US); Mike Boehmer, Melbourne, FL (US)

(21) Appl. No.: 16/938,594

(22) Filed: Jul. 24, 2020

Related U.S. Application Data
(60) Provisional application No. 62/878,509, filed on Jul. 25, 2019.

(51) Int. Cl. A61K 9/06; A61K 47/10; A61K 47/46; A61K 9/00; A61K 47/20; A61K 31/455; A61K 31/4164; A61K 31/194; A61K 31/35; A61K 47/44; A61P 17/12; A61K 8/9789; A61K 8/04; A61K 8/06; A61Q 19/08; A61P 29/00

(52) U.S. Cl. CPC A61K 9/06; A61P 17/10; A61K 47/46; A61K 9/0014; A61K 47/20; A61K 31/455; A61K 31/4164; A61K 31/194; A61K 31/35; A61K 47/44; A61P 17/12; A61K 8/9789; A61K 8/042; A61K 8/06; A61Q 19/08; A61P 29/00; A61K 47/10

(57) ABSTRACT

The invention relates to a topical composition, comprising a suitable base carrying an antioxidant component comprising turmeric or its derivative. The invention also relates to an anti-aging composition, comprising a suitable base for carrying an antioxidant component comprising: alpha lipoic acid, resveratrol, Vitamin E or its derivative, and turmeric or its derivative. The invention additionally relates to a composition for treating warts, comprising: 5-fluorouracil in an amount of about 4.5-5.5 wt %, salicylic acid in an amount of about 28-75 wt %, and a suitable base for carrying the components in the composition, in an amount of about 19.5-67.5 wt %.

### CROSS-REFERENCE TO RELATED APPLICATIONS

This application claims priority to U.S. Provisional Patent Application No. 62/878,509, filed Jul. 25, 2019, which is incorporated herein by reference in its entirety.

### FIELD OF THE INVENTION

This invention generally relates to a topical composition, an anti-aging composition, and a composition for treating warts.

### BACKGROUND OF THE INVENTION

Many active ingredients have been discovered to deliver cosmetic benefits, such as improvement in the appearance of photo-damaged or naturally aged skin, treatment of age spots, etc. However, to improve the potency of these active ingredients, high concentrations are typically used, which can cause skin irritation or inflammation. Inflammation of skins can generally be improved by inhibiting the skin penetration of certain active ingredients in the cosmetic composition by reducing the amount of active ingredients in the composition. However, such method of addressing skin inflammation impairs the efficacy of the composition. Additionally, treatments of rosacea and/or acne have also been developed by using a topical composition that can produce an anti-inflammatory effect.

The incidence of plantar warts is 1 to 2 percent in the general population, with 60% of cases resolving spontaneously within a two year period. To date, there is no uniformly effective treatment for warts, although several topical preparations are known in the art for use in the treatment of warts. Known treatments for warts typically require repeated daily applications or may not provide resolution of warts.

There thus remains a constant need in the art for developing an effective topical skin treatment composition against inflammation, acne, and/or rosacea. There also remains a need in the art for an effective anti-aging composition. There remains an additional need in the art to develop an effective treatment for warts. This invention answers those needs.

### SUMMARY OF THE INVENTION

One aspect of the invention relates to a topical composition, comprising a suitable base carrying an antioxidant component comprising turmeric or its derivative.

Another aspect of the invention relates to an anti-aging composition, comprising a suitable base for carrying an antioxidant component comprising: alpha lipoic acid, resveratrol, Vitamin E or its derivative, and turmeric or its derivative.

Another aspect of the invention relates to a composition for treating warts, comprising: 5-fluorouracil in an amount of about 4.5-5.5 wt %, salicylic acid in an amount of about 28-75 wt %, and a suitable base for carrying the components in the composition, in an amount of about 19.5-67.5 wt %.

### BRIEF DESCRIPTION OF THE FIGURES

FIG. 1A depicts a patient, prior to treatment with the composition disclosed in Example 4D.

FIG. 1B depicts the patient in FIG. 1A after receiving treatment for approximately 8 weeks with the acne composition disclosed in Example 4D.

FIG. 2A depicts a patient, prior to treatment with the composition disclosed in Example 4B.

FIG. 2B depicts another portrait of the patient in FIG. 2A.

FIG. 2C depicts the patient in FIG. 2A after receiving treatment for approximately 8 weeks with the acne composition disclosed in Example 4B.

FIG. 2D depicts another portrait of the patient in FIG. 2C, after receiving treatment for approximately 8 weeks with the acne composition disclosed in Example 4B.

FIG. 3A depicts a patient, prior to treatment with the composition disclosed in Example 9.

FIG. 3B depicts the patient in FIG. 3A after receiving treatment for approximately 4 weeks with the wart paste composition disclosed in Example 9.

FIG. 4A depicts a second patient, prior to treatment with the composition disclosed in Example 9.

FIG. 4B depicts the patient in FIG. 4A after receiving treatment for approximately 4 weeks with the wart paste composition disclosed in Example 9.

### DETAILED DESCRIPTION OF THE INVENTION

One aspect of the invention relates to a topical composition, comprising a suitable base carrying an antioxidant component comprising turmeric or its derivative.

Suitable turmeric or derivative thereof may be a turmeric extract, or a curcumin compound. Exemplary turmeric or its derivative compounds are turmeric acid, curcuminoids, and tetrahydrocurcuminoids. In one embodiment, the turmeric or its derivative is tetrahydrocurcuminoids.

The topical composition comprises a suitable base, i.e., a cosmetically acceptable vehicle to act as a diluent, dispersant, or carrier for the components for the composition, so as to facilitate their distribution when the composition is applied to the skin. Selection of the suitable base depends on the type of the formulation desired.

The topical composition may be an aqueous formulation selected from the group consisting of a cream, a gel, a lotion, a solution, an ointment, a paste, a bioadhesive, and a medicated plaster.

The suitable base may comprise one or more solvents, including but not limited to, water, ethyl alcohol, isopropanol, dimethyl sulfoxide, ethoxy diglycol, propylene glycol, ethylene glycol monomethyl ether, diethylene glycol monobutyl ether, diethylene glycol monoethyl ether, acetone, and combinations thereof. Exemplary solvents are water, ethyl alcohol, ethoxy diglycol, propylene glycol, dimethyl sulfoxide, and combinations thereof.

The suitable base may also comprise one or more stabilizers, including but not limited to, a phosphonic acid derivative, or a metabisulfite. Suitable phosphonic acid derivatives include ethylenediamine tetra (methylene phosphonic acid), hexamethylenediamine tetra (methylenephosphonic acid), diethylenetriamine penta (methylenephosphonic acid), and their salts, particularly their sodium salts, such as the pentasodium salt of ethylenediamine tetra (methylene phosphonic acid). Suitable metabisulfites may be an alkaline, alkaline earth or ammonium salt of anhydrosulphurous acid. An exemplary stabilizer is sodium metabisulfite.

The topical composition may also include other conventional additives for cosmetic formulations, such as opacifiers, fragrance, colorants, gelling agents, thickening agents, surfactants, powders, and the like.

For instance, the topical composition may be a cream formulation comprising a cream base. The cream base may contain tetrahydrocurcuminoids, ethoxy diglycol or propylene glycol, an optional component of ethyl alcohol, an optional component of sodium metabisulfite, and a moisturizing cream. In some embodiments, the cream formulation comprises a cream base containing:
about 0.1-1 wt % tetrahydrocurcuminoids,
about 1-6 wt % ethoxy diglycol or propylene glycol,
about 0-9 wt % ethyl alcohol,
about 0-0.5 wt % sodium metabisulfite, and
about 83.5-98.9 wt % a moisturizing cream.
   The amounts of these components are based on the total amount of the cream base.

The moisturizing cream can be any commercially available creams that are known to one skilled in the art that have a moisturizing effect and can be combined with other skin cosmetic ingredients. The moisturizing cream for the cream base used herein typically contains a ceramide, such as ceramide 3, ceramide 6-II, ceramide 1, or combinations thereof.

For instance, the moisturizing cream for the cream base can be a CeraVe moisturizing cream. A typical CeraVe moisturizing cream contains ingredients including ceramide 3, ceramide 6-II, ceramide 1, purified water, glycerin, ceteareth-20 and cetearyl alcohol, caprylic/capric triglyceride, behentrimonium methosulfate and cetearyl alcohol, cetyl alcohol, hyaluronic acid, cholesterol, petrolatum, dimethicone, potassium phosphate, dipotassium phosphate, sodium lauroyl lactylate, disodium EDTA, phenoxyethanol, methylparaben, propylparaben, phytosphingosine, carbomer, xanthan gum. Other commercially available moisturizing creams that contain a ceramide can also be used, for instance, Aveeno moisturizing cream, Cetaphil moisturizing cream, etc.

The topical composition may be a lotion formulation comprising a lotion base. The lotion base may contain tetrahydrocurcuminoids, ethyl alcohol, an optional component of sodium metabisulfite, and a moisturizing lotion. In some embodiments, the lotion formulation comprises a lotion base containing:
about 0.1-1 wt % tetrahydrocurcuminoids,
about 5-9 wt % ethyl alcohol,
about 0-0.5 wt % sodium metabisulfite, and
about 89.5-94.9 wt % a moisturizing lotion.
   The amounts of these components are based on the total amount of the lotion base.

Similar to the moisturizing cream, the moisturizing lotion can be any commercially available lotions that are known to one skilled in the art that have a moisturizing effect and can be combined with other skin cosmetic ingredients. The moisturizing lotion for the lotion base used herein typically contains a ceramide, such as ceramide 3, ceramide 6-II, ceramide 1, or combinations thereof. For instance, the moisturizing lotion for the lotion base can be a CeraVe moisturizing lotion. A typical CeraVe moisturizing lotion contains similar ingredients as a typical CeraVe moisturizing cream, as discussed above. Other commercially available moisturizing lotions that contain a ceramide can also be used, for instance, Aveeno moisturizing lotion, Cetaphil moisturizing lotion, etc.

The topical composition may be an ointment formulation comprising an ointment base. The ointment base may contain tetrahydrocurcuminoids, ethoxy diglycol, petrolatum, and an optional component of mineral oil. In some embodiments, the ointment formulation comprises an ointment base containing:
about 0.1-1 wt % tetrahydrocurcuminoids,
about 1-5 wt % ethoxy diglycol or propylene glycol,
about 87-98.9 wt % petrolatum, and
about 0-7 wt % mineral oil.
   The amounts of these components are based on the total amount of the ointment base.

The topical composition may be a solution formulation comprising a solution base. The solution base may contain tetrahydrocurcuminoids, dimethyl sulfoxide, ethyl alcohol, and propanediol. In some embodiments, the solution formulation comprises a solution base containing:
about 0.1-1 wt % tetrahydrocurcuminoids, about 1-5 wt % dimethyl sulfoxide, about 49-50 wt % ethyl alcohol, and about 45-48.9 wt % propanediol.
   The amounts of these components are based on the total amount of the solution base.

The topical composition can be used as an anti-inflammatory composition. In some embodiments, the anti-inflammatory composition may further comprise a) clobetasol and niacinamide. Alternatively, in some embodiments, the anti-inflammatory composition may further comprise b) fluocinodide and niacinamde. Alternatively, in some embodiments, the anti-inflammatory composition may further comprise c) triamcinolone acetonide and niacinamde. Alternatively, in some embodiments, the anti-inflammatory composition may further comprise d) desonide and niacinamde.

In some embodiments, the amount of niacinamide is about 1.5-2.5 wt % of the total amount of the topical composition.

In some embodiments, clobetasol is present, and the amount of clobetasol is about 0.01-0.2 wt % of the total amount of the topical composition. In some embodiments, fluocinodide is present, and the amount of fluocinodide is about 0.01-0.2 wt % of the total amount of the topical composition. In some embodiments, triamcinolone acetonide is present, and the amount of triamcinolone acetonide is about 0.01-0.2 wt % of the total amount of the topical composition. In some embodiments, desonide is present, and the amount of desonide is about 0.01-0.2 wt % of the total amount of the topical composition.

In one embodiment, the anti-inflammatory composition comprises a) about 0.05 or 0.1 wt % clobetasol propionate and about 2 wt % niacinamide. In one embodiment, the anti-inflammatory composition comprises b) about 0.05 wt % fluocinodide and about 2 wt % niacinamde. In one embodiment, the anti-inflammatory composition comprises c) about 0.1 wt % triamcinolone acetonide and about 2 wt % niacinamde. In one embodiment, the anti-inflammatory composition comprises d) about 0.05 wt % desonide and about 2 wt % niacinamde. The amount of each of the component is based on the total amount of the topical composition.

The anti-inflammatory composition may further comprise other anti-inflammatory and/or anti-irritant agents known to one skilled in the art.

The topical composition can be used as an acne composition. In some embodiments, the acne composition may further comprise a) tretinoin and niacinamide; and optionally hyaluronic acid or a salt thereof and water. Alternatively, in some embodiments, the acne composition may further comprise b) tretinoin, niacinamide, and acelaic acid; and optionally hyaluronic acid or a salt thereof and water.

In some embodiments, the amount of niacinamide is about 1.5-2.5 wt % of the total amount of the topical composition. In some embodiments, the amount of tretinoin is about 0.01-0.2 wt % of the total amount of the topical composition. In some embodiments, acelaic acid is present, and the amount of acelaic acid is about 5-10 wt % of the total amount of the topical composition.

In some embodiments, hyaluronic acid or a salt thereof is present, and the amount of hyaluronic acid or a salt thereof is about 0.2-0.3 wt % of the total amount of the topical composition.

In one embodiment, the acne composition comprises a) about 0.025, 0.05, or 0.1 wt % tretinoin and about 2 wt % niacinamide; and optionally, about 0.25 wt % sodium hyaluronate. In one embodiment, the acne composition comprises b) about 0.025, 0.05, or 0.1 wt % tretinoin, about 2 wt % niacinamide, and about 8 wt % acelaic acid; and optionally, about 0.25 wt % sodium hyaluronate. The amount of each of the component is based on the total amount of the topical composition.

The acne composition may further comprise other anti-inflammatory agents, anti-irritant agents, and/or agents that are effective in preventing or treating acne known to one skilled in the art.

The topical composition can be used as a rosacea composition. In some embodiments, the rosacea composition may further comprise a) acelaic acid and niacinamide. Alternatively, in some embodiments, the rosacea composition may further comprise b) acelaic acid, metronidazole, and ivermectin.

In some embodiments, the amount of acelaic acid is about 10-20 wt % of the total amount of the topical composition.

In some embodiments, niacinamide is present, and the amount of niacinamide is about 1.5-2.5 wt % of the total amount of the topical composition. In some embodiments, metronidazole is present, and the amount of metronidazole is about 0.5-1.5 wt % of the total amount of the topical composition. In some embodiments, ivermectin is present, and the amount of or ivermectin is about 0.5-1.5 wt % of the total amount of the topical composition.

In one embodiment, the rosacea composition comprises a) about 15 wt % acelaic acid and about 2 wt % niacinamide. In one embodiment, the rosacea composition comprises b) about 15 wt % acelaic acid, about 1 wt % metronidazole, and about 1 wt % ivermectin. The amount of each of the component is based on the total amount of the topical composition.

The rosacea composition may further comprise other anti-inflammatory agents, anti-irritant agents, and/or agents that are effective in preventing or treating rosacea known to one skilled in the art.

Another aspect of the invention relates to an anti-aging composition, comprising a suitable base for carrying an antioxidant component comprising: alpha lipoic acid, resveratrol, Vitamin E or its derivative, and turmeric or its derivative.

Suitable turmeric or derivative thereof may be a turmeric extract, or a curcumin compound. Exemplary turmeric or its derivative compounds are turmeric acid, curcuminoids, and tetrahydrocurcuminoids. In one embodiment, the turmeric or its derivative is tetrahydrocurcuminoids.

Any types of Vitamin E known to one skilled in the art may be used herein. Suitable Vitamin E compounds include, but are not limited to, Vitamin E and its various derivatives such as Vitamin E acetate, and Vitamin E ester.

The antioxidant component may further comprise other effective antioxidant ingredients, including but are not limited to ascorbic acid (Vitamin C), catechins, (−)-epicatechins, tocopherol such as α-tocopherol and its derivative, dimethyl-amino-ethanol (DMAE), quercetin, flavonoids and mixtures thereof; antioxidants found in green tea (including but not limited to catechins and polyphenols); antioxidants found in chocolate (including but not limited to catechins and polyphenols); antioxidants found in grapes or grape skins (including but not limited to procyanidins); and unpurified extracts of green tea, natural (“undutched”) and processed chocolate, grape skins, grape juice, wine, and other natural materials rich in antioxidants. In one embodiment, the antioxidant component further comprises ascorbic acid.

The anti-aging composition comprises a suitable base, i.e., a cosmetically acceptable vehicle to act as a diluent, dispersant, or carrier for the components for the composition, so as to facilitate their distribution when the composition is applied to the skin. Selection of the suitable base depends on the type of the formulation desired.

The anti-aging composition may be an aqueous formulation selected from the group consisting of a cream, a gel, a lotion, a solution, an ointment, a paste, a bioadhesive, and a medicated plaster.

The suitable base for carrying an antioxidant component comprises one or more solvents, a moisturizing cream, and a stabilizer.

Suitable solvents include, but are not limited to, water, ethyl alcohol, isopropanol, dimethyl sulfoxide, ethoxy diglycol, propylene glycol, ethylene glycol monomethyl ether, diethylene glycol monobutyl ether, diethylene glycol monoethyl ether, acetone, and combinations thereof. Exemplary solvents are water, ethyl alcohol, ethoxy diglycol, propylene glycol, dimethyl sulfoxide, and combinations thereof.

Suitable moisturizing creams include any commercially available creams that are known to one skilled in the art that have a moisturizing effect and can be combined with other anti-aging or antioxidant ingredients. For instance, the moisturizing cream for the cream base can be a CeraVe moisturizing cream. Other commercially available moisturizing creams can also be used, for instance, Aveeno moisturizing cream, Cetaphil moisturizing cream, etc.

Suitable stabilizers include, but are not limited to, a phosphonic acid derivative, or a metabisulfite. An exemplary stabilizer is sodium metabisulfite.

The anti-aging composition may also include other conventional additives for cosmetic formulations, such as opacifiers, fragrance, colorants, gelling agents, thickening agents, surfactants, powders, and the like.

The anti-aging composition may a cream formulation comprising i) an anti-aging cream base. The anti-aging cream base may contain: alpha lipoic acid, resveratrol, Vitamin E acetate, tetrahydrocurcuminoids, ethoxy diglycol, sodium metabisulfite, and a moisturizing cream. In some embodiments, the anti-aging cream formulation comprises an anti-aging cream base containing:
about 0.1-1 wt % alpha lipoic acid,
about 0.1-0.5 wt % resveratrol,
about 0.5-1.5 wt % Vitamin E acetate,
about 0.1-1 wt % tetrahydrocurcuminoids,
about 1-10 wt % ethoxy diglycol,
about 0.1-0.5 wt % sodium metabisulfite, and
about 85.5-98.1 wt % a moisturizing cream.
   The amounts of these components are based on the total amount of the anti-aging cream base.

In some embodiments, the anti-aging composition further comprises ii) niacinamide, iii) tretinoin, and optionally iv) hyaluronic acid or a salt thereof.

In some embodiments, the anti-aging composition comprises: i) an anti-aging cream base comprising: alpha lipoic acid, resveratrol, Vitamin E acetate, tetrahydrocurcuminoids, ethyl alcohol, ethoxy diglycol or propylene glycol, sodium metabisulfite, and a moisturizing cream; ii) niacinamide; iii) tretinoin; and optionally iv) hyaluronic acid or a salt thereof, and water.

In some embodiments, the amount of niacinamide is about 1.5-2.5 wt % of the total amount of the anti-aging composition.

In some embodiments, the amount of tretinoin is about 0.01-0.1 wt % of the total amount of the anti-aging composition.

In some embodiments, component iv) is present, and the amount of hyaluronic acid or a salt thereof is about 0.2-0.3 wt % of the total amount of the anti-aging composition. In one embodiment, hyaluronic acid or a salt thereof is sodium hyaluronate, and the amount of sodium hyaluronate is about 0.25 wt % of the total amount of the anti-aging composition.

In some embodiments, the amount of niacinamide is about 2 wt % of the total amount of the anti-aging composition; and the amount of tretinoin is about 0.0125 wt %, 0.025 wt %, or 0.05 wt % of the total amount of the anti-aging composition.

Another aspect of the invention relates to a composition for treating warts, comprising: 5-fluorouracil in an amount of about 4.5-5.5 wt %, salicylic acid in an amount of about 28-75 wt %, and a suitable base for carrying the components in the composition, in an amount of about 19.5-67.5 wt %.

The composition for treating warts comprises a suitable base, i.e., a cosmetically acceptable vehicle to act as a diluent, dispersant, or carrier for the components for the composition, so as to facilitate their distribution when the composition is applied to the skin. Selection of the suitable base depends on the type of the formulation desired.

The composition for treating warts may be an aqueous formulation selected from the group consisting of a cream, a gel, a lotion, a solution, an ointment, a paste, a bioadhesive, and a medicated plaster.

For instance, the composition may be a solution formulation. Because 5-fluoruacil may be degraded by oxidation, the solution formulation typically is stabilized by having a pH of 8 or above.

The suitable base may comprise one or more solvents, for instance, a binary solvent system containing a mixture of two immiscible solvents. In one embodiment, the suitable base comprises a binary solvent system containing water and triethanolamine, which is used to control the pH of the solution formation.

The suitable base may also comprise one or more stabilizers, including but not limited to, a phosphonic acid derivative, or a metabisulfite. An exemplary stabilizer is sodium metabisulfite.

In some embodiments, the solution formulation for treating warts comprises 5-fluorouracil, salicylic acid, water, triethanolamine, and optionally, sodium metabisulfite. In one embodiment, the solution formulation for treating warts comprises:
about 4.5-5 wt % 5-fluorouracil,
about 28-30 wt % salicylic acid,
about 27.5-37.5 wt % water,
about 30-37 wt % triethanolamine, and
about 0-0.5 wt % sodium metabisulfite.

The composition for treating warts may also be an ointment or paste formulation.

The suitable base for the ointment or paste formulation may comprise solvents containing water, triethanolamine, and dimethyl sulfoxide.

The suitable base may further comprise at least one emulsifiable base and an emulsifier.

Suitable emulsifiable or emulsion bases, also referred to as absorbent ointment bases, may contain little or no water and may include, for example, hydroxystearin sulfate, lanolin or anhydrous lanolin, petrolatum, cetyl alcohol, glyceryl monostearate, stearic acid, and combinations thereof. An exemplary emulsifiable base is lanolin.

Suitable emulsifiers include any emulsifiers known to one skilled in the art that can be used together with an emulsifiable or emulsion base (i.e., an oil or oily material) to provide a water-in-oil emulsion or an oil-in-water-emulsion. An exemplary emulsifier is polysorbate, e.g., polysorbate 20.

In one embodiment, the suitable base includes lanolin and polysorbate 20.

In some embodiments, the ointment or paste formulation for treating warts comprises 5-fluorouracil, salicylic acid, water, dimethyl sulfoxide, triethanolamine, lanolin, and polysorbate (e.g., polysorbate 20). In one embodiment, the ointment or paste formulation for treating warts comprises:
about 4.5-5 wt % 5-fluorouracil,
about 65-70 wt % salicylic acid,
about 2-8.5 wt % water,
about 2-3 wt % dimethyl sulfoxide,
about 7-9 wt % triethanolamine,
about 6-8 wt % lanolin, and
about 7-9 wt % polysorbate (e.g., polysorbate 20).

### EXAMPLES

The following examples are for illustrative purposes only and are not intended to limit, in any way, the scope of the present invention.

### Example 1A—A Turmeric Base for a Topical Composition—a Cream Vehicle

TABLE 1
A cream vehicle for a turmeric base

| | | Ethoxy | CeraVe | Sodium |
|---|---|---|---|---|
| | THC | diglycol | moisturing | metabisulfite |
| Ingredient | powder | liquid | cream | NF granule |
| Quantity (gms) | 0.5 | 4 | 95.3 | 0.2 |

Manufacturing Procedure:
1. Tetrahydrocurcuminoids (THC) was weighed into a weigh boat and added to a mortar.
2. The triturate ingredients in mortar were dried with pestle to achieve a fine white powder mixture with uniform particle size.
3. Ethoxy diglycol was weighed into a syringe.
4. The dry ingredients in the mortar were wetted with ethoxy diglycol to form a white paste or slurry.
5. CeraVe cream was weighed into a weigh boat or other tared vessel, and was added to the paste/slurry in the mortar using geometric dilution.
6. The mixture in the mortar was mixed to create a white homogenous mixture, and transferred into an appropriately-sized Unguator® jar for further mixing for 2 cycles (2 minutes, twice).
7. The formulation was obtained as a white cream with a shiny surface.

A lotion vehicle was similarly prepared by replacing the moisturizing cream with a moisturizing lotion.

### Example 1B—A Turmeric Base for a Topical Composition—a Solution Vehicle

TABLE 2
A solution vehicle for a turmeric base

| | THC | Ethyl alcohol | Dimethyl sulfoxide | Propanediol |
|---|---|---|---|---|
| Ingredient | powder | USP liquid | USP liquid | liquid |
| Quantity (gms) | 0.5 | 50 | 3 | 46.5 |

Manufacturing Procedure:
1. THC and sodium metabisulfite were weighed into weigh boats and added to a mortar.
2. Dimethyl sulfoxide (DMSO) was weighed into a syringe. Ethanol and propanediol were weighed into small beakers. All the liquids were added in an appropriately-sized batch beaker equipped with a stir bar, which was then covered tightly with press and seal wrap.
3. The batch beaker was placed on a CIMAREK hotplate/stir device (Medline Industries, Inc.), and was stirred without heating.
4. THC was added to the rapidly spinning solution in the batch beaker until complete dissolution occurred.
5. When the solution was completely clear, visual particulate test was performed using black/white background to assure complete dissolution of solids.
6. The formulation was obtained as a clear, slightly viscous liquid.

### Example 1C—A Turmeric Base for a Topical Composition—an Ointment Vehicle

TABLE 3
An ointment vehicle for a turmeric base

| | THC | Ethoxy | Petrolatum white |
|---|---|---|---|
| Ingredient | powder | diglycol liquid | USP |
| Quantity (gms) | 0.5 | 2 | 97.5 |

Manufacturing Procedure:
1. THC was weighed into a weigh boat and added to a mortar.
2. The triturate ingredients in mortar were dried with pestle to achieve a fine white powder mixture with uniform particle size.
3. Ethoxy diglycol was weighed into a syringe.
4. The dry ingredients in the mortar were wetted with ethoxy diglycol to form a white paste or slurry.
5. Petrolatum was weighed into a weigh boat or other tared vessel, and was added to the paste/slurry in the mortar using geometric dilution.
6. The mixture in the mortar was mixed to create a white homogenous mixture, and transferred into an appropriately-sized Unguator® jar for further mixing for 2 cycles (2 minutes, twice).
7. The formulation was obtained as a translucent, off-white ointment.

### Example 2—Manufacture of an Aqueous Formulation (Cream, Lotion, Solution, or Ointment)

In this example, various ingredients were combined with the turmeric base prepared according to Example 1A, Example 1B, or Example 1C, depending on the type of formulation desired, to prepare a topical composition (anti-inflammatory compositions in Example 3, acne compositions in Example 4, rosacea compositions in Example 5).

A typical manufacturing procedure for a cream formulation:
1. The dry ingredients were weighed into individual weigh boats and combined in an appropriately-sized glass mortar.
2. The triturate ingredients in mortar were dried with pestle to achieve a fine white powder mixture with uniform particle size.
3. The solvent(s) was weighed using a syringe.
4. The dry ingredients in the mortar were wetted with the solvent(s) to form a thick, smooth paste or slurry.
5. The cream vehicle (e.g., prepared according to Example 1A) was weighed into separate weigh boats, and was added to the paste/slurry in the mortar using geometric dilution.
6. The mixture in the mortar was mixed to create a white homogenous mixture, and transferred into an appropriately-sized Unguator® jar for further mixing for 2 cycles (2 minutes, twice), resulting in a cream. Between the two mixing cycles, a visual grit test was performed and, when needed, the mixture can be processed through an ointment mill before the second mixing cycle.

A typical manufacturing procedure for a lotion formulation is the same as the typical manufacturing procedure for a cream formulation, except that the cream vehicle was replaced with a lotion vehicle.

A typical manufacturing procedure for a solution formulation:
1. The dry ingredients were weighed into individual weigh boats.
2. The solution vehicle (e.g., prepared according to Example 1B) was weighed into an appropriately-sized beaker equipped with a stir bar, which was then covered tightly with press and seal wrap.
3. The dry ingredients were added into the beaker, and the beaker was placed on the CIMAREK device and was stirred without heating until the solution was completely clear.

A typical manufacturing procedure for an ointment formulation:
1. The dry ingredients were weighed into individual weigh boats and combined in an appropriately-sized glass mortar.
2. The triturate ingredients in mortar were dried with pestle to achieve a fine white powder mixture with uniform particle size.
3. The mineral oil was weighed using a syringe.
4. The dry ingredients in the mortar were wetted with the mineral oil to form a white slurry.
5. The ointment vehicle (e.g., prepared according to Example 1C) was weighed into separate weigh boats, and was added to the paste/slurry in the mortar using geometric dilution.
6. The mixture in the mortar was mixed to create a homogenous mixture, and transferred into an appropriately-sized Unguator® jar for further mixing for 2 cycles (2 minutes, twice), resulting in an ointment. Between the two mixing cycles, a visual grit test was performed and, when needed, the mixture can be processed through an ointment mill before the second mixing cycle.

### Example 3—Anti-Inflammatory Compositions

In this example, various anti-inflammatory compositions were prepared using the turmeric base prepared according to Example 1A, Example 1B, or Example 1C, following the manufacturing procedures (depending on whether it was a cream, lotion, solution, or ointment formulation) according to Example 2.

### Example 3A—Clobetasol 0.05%, Niacinamide 2%

TABLE 4
An anti-inflammatory cream

| | Clobetasol | | | Ethyl |
|---|---|---|---|---|
| | propionate | Niacinamide | Cream vehicle | alcohol |
| Ingredient | USP micronized | USP powder | (Example IA) | USP liquid |
| Quantity | 0.05 | 2 | 92.95 | 5 |
| (gms) | | | | |

TABLE 5
An anti-inflammatory solution

| | Clobetasol propionate | Niacinamide | Solution vehicle |
|---|---|---|---|
| Ingredient | USP micronized | USP powder | (Example 1B) |
| Quantity (gms) | 0.05 | 2 | 97.95 |

TABLE 6
An anti-inflammatory ointment

| | Clobetasol | | Ointment | |
|---|---|---|---|---|
| | propionate | Niacinamide | vehicle | Mineral oil |
| Ingredient | USP micronized | USP powder | (Example 1C) | NF (light) |
| Quantity | 0.05 | 2 | 94.95 | 3 |
| (gms) | | | | |

The following chart shows the results of treating representative patients with the anti-inflammatory cream/solution/ointment of this example.

| | | | | cream/ |
|---|---|---|---|---|
| | | | Score at | solution/ |
| Patient | Demographics | Score at Baseline | Follow up | ointment |
| Patient | 15-20 year old | 3 - moderate to severe | 1 - mild | cream |
| 1 | male | erythema, scaliness | scaling | |
| | | for eczema | and flaking | |
| Patient | 25-30 year old | 2 - moderate erythema | 0 - clear | cream |
| 2 | female | and scaling for | | |
| | | eczema | | |
| Patient | 40-45 year old | 3 - moderate to severe | 1 - mild | cream |
| 3 | female | erythema, scaliness | scaling | |
| | | for hand eczema | and flaking | |
| Patient | 25-30 year old | 2 - moderate erythema | 0 - clear | cream |
| 4 | female | and scaling for | | |
| | | eczema | | |
| Patient | 45-50 year old | 3 - moderate to severe | 1 - mild | solution |
| 5 | female | psoriasis thick scales | scaling | |
| | | on scalp | and flaking | |

### Example 3B—Clobetasol 0.1%, Niacinamide 2%

TABLE 7
An anti-inflammatory cream

| | Clobetasol | | | |
|---|---|---|---|---|
| | propionate | Niacinamide | Cream vehicle | Ethyl alcohol |
| Ingredient | USP micronized | USP powder | (Example 1A) | USP liquid |
| Quantity | 0.1 | 2 | 92.9 | 5 |
| (gms) | | | | |

TABLE 8
An anti-inflammatory solution

| | Clobetasol propionate | Niacinamide | Solution vehicle |
|---|---|---|---|
| Ingredient | USP micronized | USP powder | (Example 1B) |
| Quantity (gms) | 0.1 | 2 | 99.9 |

TABLE 9
An anti-inflammatory ointment

| | Clobetasol | | Ointment | |
|---|---|---|---|---|
| | propionate | Niacinamide | vehicle | Mineral oil |
| Ingredient | USP micronized | USP powder | (Example 1C) | NF (light) |
| Quantity | 0.1 | 2 | 92.9 | 5 |
| (gms) | | | | |

The following chart shows the results of treating representative patients with the anti-inflammatory cream/solution/ointment of this example.

| | | | | cream/ |
|---|---|---|---|---|
| | | | Score at | solution/ |
| Patient | Demographics | Score at Baseline | Follow up | ointment |
| Patient 1 | 60-65 year old | 4 - severe redness, | 0 - clear | cream |
| | male | scaliness, and | | |
| | | excoriation for | | |
| | | eczema | | |
| Patient 2 | 40-45 year old | Psoriasis 3 - moderate | 1 - mild | ointment |
| | female | to severe thick scales | scaling | |
| | | | and flaking | |
| Patient 3 | 30-35 year old | 4 - severe redness, | 2-moderate | cream |
| | female | scaliness, and | erythema | |
| | | excoriation for | left over, | |
| | | eczema | flaking | |
| | | | minimal | |
| Patient 4 | 40-45 year old | 4 - psoriasis thick | 1 - mild | ointment |
| | female | plaques on hands and | scaling | |
| | | feet | and flaking | |
| Patient 5 | 30-35 year old | 3 - eczmea moderate to | 0 - clear | cream |
| | male | severe scaling and | | |
| | | erythema | | |
| Patient 6 | 50-55 year old | 4 - severe redness, | 1 - mild | cream |
| | male | scaliness, and | scaling | |
| | | excoriation for | and flaking | |
| | | eczema | | |
| Patient 7 | 55-60 year old | 4 - severe redness, | 0 - clear | cream |
| | male | scaliness, and | | |
| | | excoriation for | | |
| | | eczema | | |
| Patient 8 | 50-55 year old | 3 - eczmea moderate to | 0 - clear | cream |
| | male | severe scaling and | | |
| | | erythema | | |
| Patient 9 | 65-70 year old | 4 - severe redness, | 1 - mild | cream |
| | male | scaliness, and | scaling | |
| | | excoriation for | and flaking | |
| | | eczema | | |
| Patient | 30-35 year old | 3 - eczmea moderate to | 0 - clear | cream |
| 10 | female | severe scaling and | | |
| | | erythema | | |

### Example 3C—Fluocinonide 0.05%, Niacinamide 2%

TABLE 10
An anti-inflammatory cream

| | Ingredient | | | |
|---|---|---|---|---|
| | Fluocinonide | Niacinamide | Cream vehicle | Ethyl alcohol |
| | USP micronized | USP powder | (Example 1A) | USP liquid |
| Quantity | 0.05 | 2 | 92.95 | 5 |
| (gms) | | | | |

TABLE 11
An anti-inflammatory solution

| | Ingredient | | |
|---|---|---|---|
| | Fluocinonide | Niacinamide | Solution vehicle |
| | USP micronized | USP powder | (Example 1B) |
| Quantity (gms) | 0.05 | 2 | 97.95 |

TABLE 12
An anti-inflammatory ointment

| | Ingredient | | | |
|---|---|---|---|---|
| | Fluocinonide | Niacinamide | Ointment vehicle | Mineral oil |
| | USP micronized | USP powder | (Example 1C) | NF (light) |
| Quantity | 0.05 | 2 | 92.95 | 5 |
| (gms) | | | | |

The following chart shows the results of treating representative patients with the anti-inflammatory cream/solution/ointment of this example.

| | Demo- | | Score at | cream/solution/ |
|---|---|---|---|---|
| Patient | graphics | Score at Baseline | Follow up | ointment |
| Patient | 45-50 year | 3-eczema | 1-mild erythema | cream |
| 1 | old female | moderate to severe | left over | |
| Patient | 50-55 year | 3-eczema | 1-mild erythema | ointment |
| 2 | old male | moderate to severe | left over | |
| Patient | 50-55 year | 2-eczema | 1-mild erythema | cream |
| 3 | old female | moderate | left over | |

TABLE 13
An anti-inflammatory cream

| | Ingredient | | | |
|---|---|---|---|---|
| | Triamcinolone | | | Ethyl |
| | acetonide | Niacinamide | Cream vehicle | alcohol |
| | USP micronized | USP powder | (Example 1A) | USP liquid |
| Quantity | 0.1 | 2 | 92.9 | 5 |
| (gms) | | | | |

### Example 3D—Triamcinolone Acetonide 0.1%, Niacinamide 2%

TABLE 14
An anti-inflammatory solution

| | Ingredient | | |
|---|---|---|---|
| | Triamcinolone acetonide | Niacinamide | Solution vehicle |
| | USP micronized | USP powder | (Example 1B) |
| Quantity (gms) | 0.1 | 2 | 97.9 |

TABLE 15
An anti-inflammatory ointment

| | Ingredient | | | |
|---|---|---|---|---|
| | Triamcinolone | | Ointment | |
| | acetonide USP | Niacinamide | vehicle | Mineral oil |
| | micronized | USP powder | (Example 1C) | NF (light) |
| Quantity (gms) | 0.1 | 2 | 92.9 | 5 |

The following chart shows the results of treating representative patients with the anti-inflammatory cream/solution/ointment of this example.

| | Demo- | | Score at | cream/solution/ |
|---|---|---|---|---|
| Patient | graphics | Score at Baseline | Follow up | ointment |
| Patient 1 | 60-65 year | 3-moderate to severe | 0-clear | cream |
| | old male | seborrheic dermatitis | | |
| | | flaking and erythema | | |

### Example 3E—Desonide 0.05%, Niacinamide 2%

TABLE 16
An anti-inflammatory cream

| | Ingredient | | | |
|---|---|---|---|---|
| | Desonide USP | Niacinamide | Cream vehicle | Ethyl alcohol |
| | micronized | USP powder | (Example 1A) | USP liquid |
| Quantity | 0.05 | 2 | 92.95 | 5 |
| (gms) | | | | |

TABLE 17
An anti-inflammatory ointment

| | Ingredient | | | |
|---|---|---|---|---|
| | Desonide USP | Niacinamide | Ointment vehicle | Mineral oil |
| | micronized | USP powder | (Example 1C) | NF (light) |
| Quantity | 0.05 | 2 | 92.95 | 5 |
| (gms) | | | | |

TABLE 18
An anti-inflammatory lotion

| | Ingredient | | | | |
|---|---|---|---|---|---|
| | Desonide | | | Ethyl | CeraVe |
| | USP | Niacinamide | THC | alcohol | moisturing |
| | micronized | USP powder | powder | USP liquid | lotion |
| Quantity | 0.05 | 2 | 0.5 | 7 | 90.45 |
| (gms) | | | | | |

The following chart shows the results of treating representative patients with the anti-inflammatory cream/solution/ointment of this example.

| | | | Score at | cream/ |
|---|---|---|---|---|
| | Demo- | | Follow | solution/ |
| Patient | graphics | Score at Baseline | up | ointment |
| Patient 1 | 60-65 year | 4-severe erythema and | 0-clear | cream |
| | old male | flaking in and around | | |
| | | ears seb dermatitis | | |
| Patient 2 | 50-55 year | 3-moderate to severe | 1-mild | cream |
| | old female | flaking and erythema in ears | flaking | |
| Patient 3 | 35-40 year | 3-moderate to severe | 0-clear | cream |
| | old male | flaking and erythema in ears | | |
| Patient 4 | 30-35 year | 2-moderate flaking | 0-clear | cream |
| | old female | and erythema in facial folds | | |
| Patient 5 | 40-45 year | 3-moderate to severe | 1-mild | cream |
| | old male | flaking and erythema | flaking | |
| | | in eyebrows and NLF | | |
| Patient 6 | 45-50 year | 3-moderate to severe | 0-clear | cream |
| | old female | flaking and erythema in NLF | | |
| Patient 7 | 55-60 year | 3-moderate to severe | 1-mild | cream |
| | old male | flaking and erythema | flaking | |
| | | in and around ears | | |
| Patient 8 | 50-55 year | 3-moderate to severe flaking | 0-clear | cream |
| | old male | and erythema in eyebrows | | |
| Patient 9 | 25-30 year | 2-moderate flaking | 0-clear | cream |
| | old male | and erythema in eyebrows | | |
| Patient 10 | 40-45 year | 3-moderate to severe | 0-clear | cream |
| | old female | flaking and erythema in NLF | | |

### Example 4—Acne Compositions

In this example, various acne compositions were prepared using the turmeric base prepared according to Example 1A, Example 1B, or Example 1C, following the manufacturing procedures (depending on whether it was a cream, lotion, solution, or ointment formulation) according to Example 2.

For the treatment results using the acne compositions in this example, below is the acne grading scores that are referenced in the charts.

| GRADE | VALUE | DEFINITION |
|---|---|---|
| Clear | 0 | Normal, clear skin with no evidence of acne vulgaris |
| Almost | 1 | Rare noninflammatory lesions present, with rare |
| clear | | noninflamed papules (papules must be resolving and |
| | | may be hyperpigmented. though not pink-red) |
| Mild | 2 | Some noninflammatory lesions are present, with few |
| | | inflammatory lesions (papules/pustules only, no |
| | | nodulocystic lesions) |
| Moderate | 3 | Nonintlammatory lesions predominate, with multiple |
| | | inflammatory lesions evident: several to many |
| | | comedones and papules/pustules: there may or may |
| | | not be one small nodulocystic lesion |
| Severe | 4 | Inflammatory lesions are more apparent, many |
| | | comedones and papules/pustules, there may or |
| | | may not be a few nodulocystic lesions |
| Very | 5 | Highly inflammatory lesions predominate, variable |
| severe | | number of comedones. many papules/pustules. and |
| | | many nodulocystic lesions |

From: Schlessinger J, Menter A, Gold M, et al. Clinical safety and efficacy studies of a novel formulation combining 1.2% clindamycin phosphate and 0.025% tretinoin for the treatment of acne vulga

### Example 4A—Tretinoin (0.025%, 0.05%, 0.1%), Niacinamide 2%

TABLE 19
An acne cream

| | Ingredient | | | |
|---|---|---|---|---|
| | Tretinoin | Niacinamide | Cream vehicle | Ethyl alcohol |
| | USP powder | USP powder | (Example 1A) | USP liquid |
| Quantity | 0.025 | 2 | 92.975 | 5 |
| (gms) | | | | |

TABLE 20
An acne cream

| | Ingredient | | | |
|---|---|---|---|---|
| | Tretinoin USP | Niacinamide | Cream vehicle | Ethyl alcohol |
| | powder | USP powder | (Example IA) | USP liquid |
| Quantity | 0.05 | 2 | 92.95 | 5 |
| (gms) | | | | |

TABLE 21
An acne cream

| | Tretinoin | Niacinamide | Cream vehicle | Ethyl alcohol |
|---|---|---|---|---|
| Ingredient | USP powder | USP powder | (Example 1A) | USP liquid |
| Quantity (gms) | 0.1 | 2 | 92.9 | 5 |

### Example 4B—Tretinoin (0.025%, 0.05%, 0.1%), Niacinamide 2%, Sodium Hyaluronate 0.25%

TABLE 22
An acne cream

| | Ingredient | | | | | |
|---|---|---|---|---|---|---|
| | Tretinoin | | | Ethyl | Sodium | Sterile |
| | USP | Niacinamide | Cream vehicle | alcohol | Hyaluronate | water USP |
| | powder | USP powder | (Example 1A) | USP liquid | powder | liquid |
| Quantity (gms) | 0.025 | 2 | 80.725 | 6 | 0.25 | 11 |

TABLE 23
An acne cream

| | Ingredient | | | | | |
|---|---|---|---|---|---|---|
| | Tretinoin | | | Ethyl | Sodium | Sterile |
| | USP | Niacinamide | Cream vehicle | alcohol | Hyaluronate | water USP |
| | powder | USP powder | (Example 1A) | USP liquid | powder | liquid |
| Quantity (gms) | 0.05 | 2 | 80.7 | 7 | 0.25 | 11 |

TABLE 24
An acne cream

| | Ingredient | | | | | |
|---|---|---|---|---|---|---|
| | Tretinoin | | | Ethyl | Sodium | Sterile |
| | USP | Niacinamide | Cream vehicle | alcohol | Hyaluronate | water USP |
| | powder | USP powder | (Example 1A) | USP liquid | powder | liquid |
| Quantity (gms) | 0.1 | 2 | 80.65 | 7 | 0.25 | 11 |

### Example 4C—Tretinoin (0.025%, 0.05%, 0.1%), Niacinamide 2%, Azelaic Acid 8%

TABLE 25
An acne cream

| | Ingredient | | | | | | |
|---|---|---|---|---|---|---|---|
| | Tretinoin | | Azelaic | | Ethyl | Propylene | CeraVe |
| | USP | Niacinamide | acid flakes | THC | alcohol | glycol USP | moisturing |
| | powder | USP powder | powder | powder | USP liquid | solution | cream |
| Quantity | 0.025 | 2 | 8 | 0.5 | 7 | 5 | 77.475 |
| (gms) | | | | | | | |

TABLE 26
An acne cream

| | Ingredient | | | | | | |
|---|---|---|---|---|---|---|---|
| | Tretinoin | | Azelaic | | Ethyl | Propylene | CeraVe |
| | USP | Niacinamide | acid flakes | THC | alcohol | glycol USP | moisturing |
| | powder | USP powder | powder | powder | USP liquid | solution | cream |
| Quantity | 0.05 | 2 | 8 | 0.5 | 7 | 5 | 77.45 |
| (gms) | | | | | | | |

TABLE 27
An acne cream

| | Ingredient | | | | | | |
|---|---|---|---|---|---|---|---|
| | Tretinoin | | Azelaic | | Ethyl | Propylene | CeraVe |
| | USP | Niacinamide | acid flakes | THC | alcohol | glycol USP | moisturing |
| | powder | USP powder | powder | powder | USP liquid | solution | cream |
| Quantity | 0.1 | 2 | 8 | 0.5 | 7 | 5 | 77.4 |
| (gms) | | | | | | | |

### Example 4D—Tretinoin (0.025%, 0.05%, 0.1%), Niacinamide 2%, Azelaic Acid 8%, Sodium Hyaluronate 0.25%

TABLE 28
An acne cream

| | Tretinoin | Niacinamide | Azelaic acid | THC | Ethyl alcohol |
|---|---|---|---|---|---|
| Ingredient | USP powder | USP powder | flakes powder | powder | USP liquid |
| Quantity (gms) | 0.025 | 2 | 8 | 0.5 | 7 |
| Ingredient | Propylene | CeraVe | Sodium | Sterile water | Sodium |
| | glycol USP | moisturing | Hyaluronate | USP liquid | metabisulfite NF |
| | solution | cream | powder | | granule |
| Quantity (gms) | 5 | 72.25 | 0.25 | 5 | 0.2 |

TABLE 29
An acne cream

| | Tretinoin | Niacinamide | Azelaic acid | THC | Ethyl alcohol |
|---|---|---|---|---|---|
| Ingredient | USP powder | USP powder | flakes powder | powder | USP liquid |
| Quantity (gms) | 0.05 | 2 | 8 | 0.5 | 7 |
| Ingredient | Propylene | CeraVe | Sodium | Sterile water | Sodium |
| | glycol USP | moisturing | Hyaluronate | USP liquid | metabisulfite NF |
| | solution | cream | powder | | granule |
| Quantity (gms) | 5 | 72.28 | 0.25 | 5 | 0.2 |

TABLE 30
An acne cream

| | Tretinoin | Niacinamide | Azelaic acid | THC | Ethyl alcohol |
|---|---|---|---|---|---|
| Ingredient | USP powder | USP powder | flakes powder | powder | USP liquid |
| Quantity (gms) | 0.1 | 2 | 8 | 0.5 | 7 |
| Ingredient | Propylene | CeraVe | Sodium | Sterile water | Sodium |
| | glycol USP | moisturing | Hyaluronate | USP liquid | metabisulfite NF |
| | solution | cream | powder | | granule |
| Quantity (gms) | 5 | 71.95 | 0.25 | 5 | 0.2 |

The following chart shows the results of treating representative patients with the acne cream/solution/ointment of this example.

| | | | | |
|---|---|---|---|---|
| | With or without | | | |
| | Hyaluronic | | Score at | Score at End |
| Patient | Acid | Demographics | Baseline | of Treatment |

Tretinoin 0.025%, Niacinamide 2%, Cream

| | | | | |
|---|---|---|---|---|
| Patient 1* | With HA | 25-30 y/o male | 4 | 2 |
| Patient 2 | With HA | 30-35 year old female | 2 | 1 |
| Patient 3 | With HA | 20-25 year old female | 2 | 0 |
| Patient 4 | With HA | 15-20 year old female | 2 | 1 |
| Patient 5 | With HA | 15-20 year old male | 3 | 1 |
| Patient 6 | With HA | 10-15 year old female | 2 | 1 |

Tretinoin 0.05%, Niacinamide 2%, Cream

| | | | | |
|---|---|---|---|---|
| Patient 1 | with HA | 15-20 year old female | 4 | 2 |
| Patient 2 | with HA | 10-15 year old female | 3 | 1 |
| Patient 3 | with HA | 10-15 year old male | 4 | 1 |
| Patient 4 | with HA | 15-20 year old male | 4 | 3 |
| Patient 5 | with HA | 20-25 year old female | 3 | 0 |
| Patient 6 | with HA | 10-15 year old female | 2 | 0 |
| Patient 7 | with HA | 30-35 year old female | 5 | 3 |
| Patient 8 | with HA | 25-30 year old male | 3 | 1 |
| Patient 9 | with HA | 15-20 year old male | 4 | 1 |
| Patient 10 | with HA | 15-20 year old female | 4 | 2 |

Tretinoin 0.1%, Niacinamide 2%, Cream

| | | | | |
|---|---|---|---|---|
| Patient 1 | with HA | 15-20 year old male | 5 | 2 |
| Patient 2 | with HA | 10-15 year old male | 4 | 1 |
| Patient 3 | with HA | 20-25 year old female | 5 | 3 |
| Patient 4 | with HA | 10-15 year old male | 4 | 1 |
| Patient 5 | with HA | 15-20 year old female | 5 | 1 |
| Patient 6 | with HA | 15-20 year old female | 5 | 2 |
| Patient 7 | with HA | 10-15 year old female | 4 | 1 |
| Patient 8 | with HA | 15-20 year old male | 5 | 2 |
| Patient 9 | with HA | 10-15 year old male | 4 | 2 |
| Patient 10 | with HA | 25-30 year old female | 5 | 3 |

Tretinoin 0.025%, Niacinamide 2%, Azelaic Acid 8%, Cream

| | | | | |
|---|---|---|---|---|
| Patient 1 | with HA | 25-30 year old female | 2 | 1 |
| Patient 2 | with HA | 30-35 year old male | 2 | 1 |

Tretinoin 0.05%, Niacinamide 2%, Azelaic Acid 8%, Cream

| | | | | |
|---|---|---|---|---|
| Patient 1** | with HA | 30-35 year old female | 2 | 1 |
| Patient 2 | with HA | 15-20 year old female | 4 | 2 |
| Patient 3 | with HA | 15-20 year old female | 3 | 1 |
| Patient 4 | with HA | 25-30 year old female | 4 | 2 |
| Patient 5 | with HA | 15-20 year old male | 4 | 2 |

Tretinoin 0.1%, Niacinamide 2%, Azelaic Acid 8%, Cream

| | | | | |
|---|---|---|---|---|
| Patient 1 | with HA | 20-25 year old female | 5 | 2 |
| Patient 2 | with HA | 15-20 year old female | 5 | 3 |
| Patient 3 | with HA | 15-20 year old male | 5 | 2 |
| Patient 4 | with HA | 20-25 year old female | 4 | 2 |
| Patient 5 | with HA | 10-15 year old female | 4 | 1 |
| Patient 6 | with HA | 15-20 year old female | 4 | 0 |
| Patient 7 | without HA | 15-20 year old male | 4 | 3 |
| Patient 8 | with HA | 25-30 year old female | 5 | 2 |
| Patient 9 | with HA | 20-25 year old male | 4 | 2 |
| Patient 10 | with HA | 15-20 year old male | 4 | 0 |

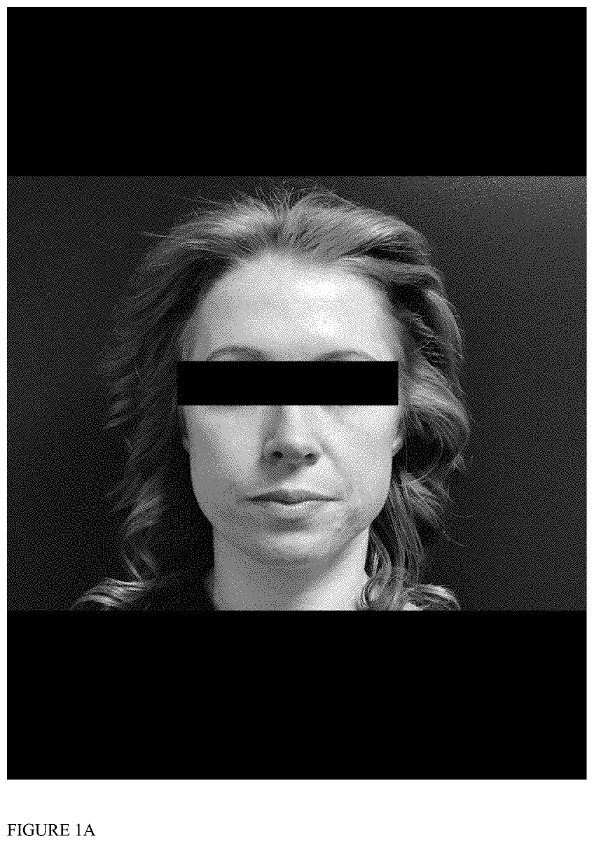

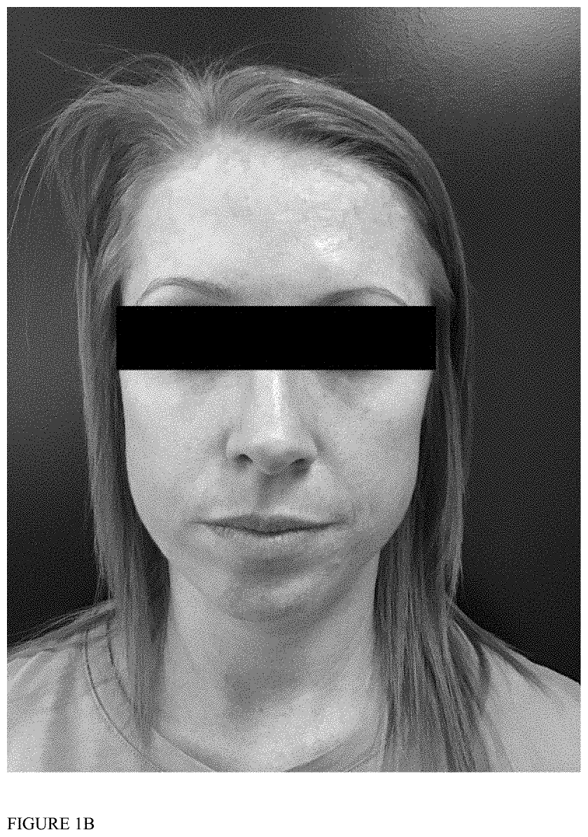

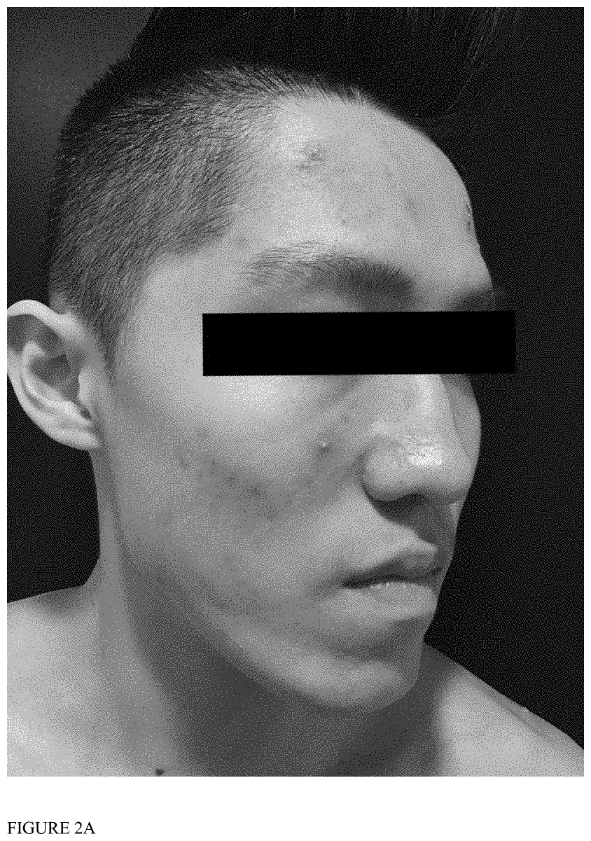

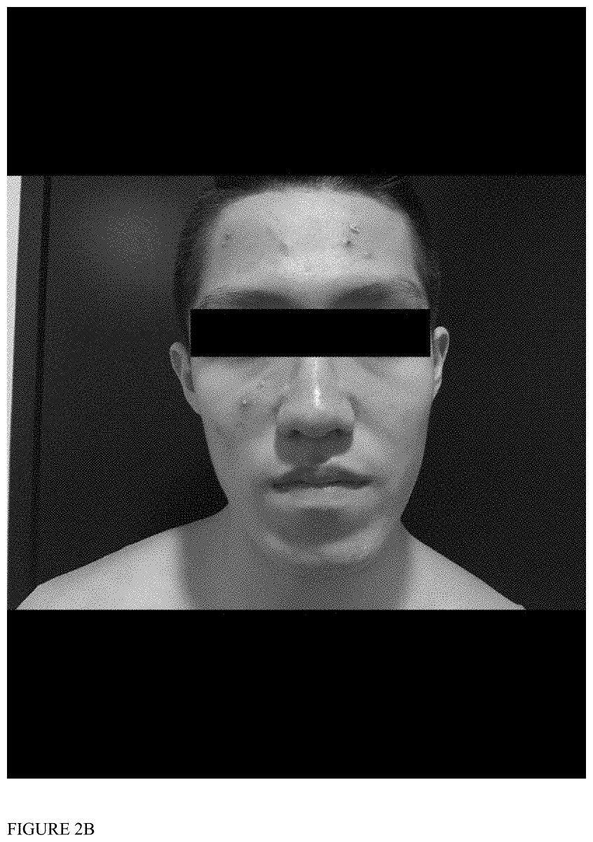

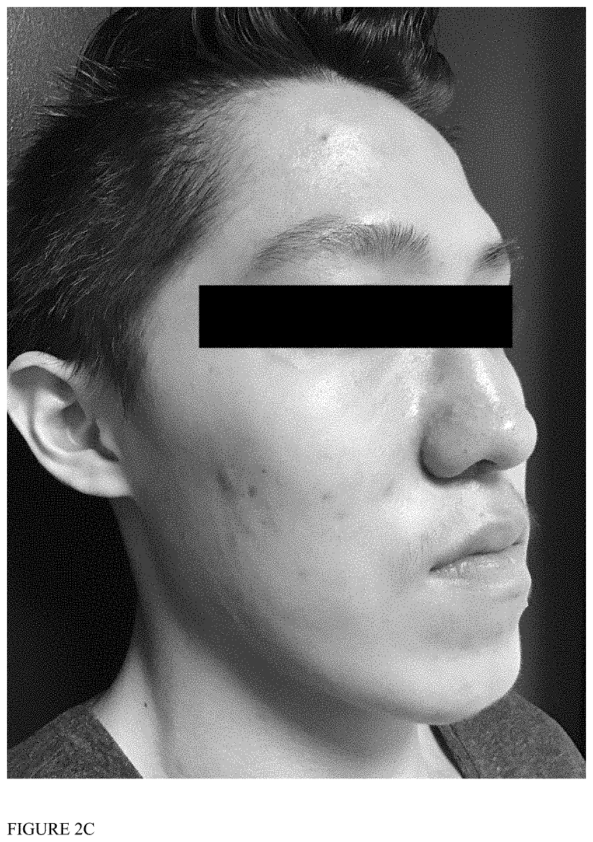

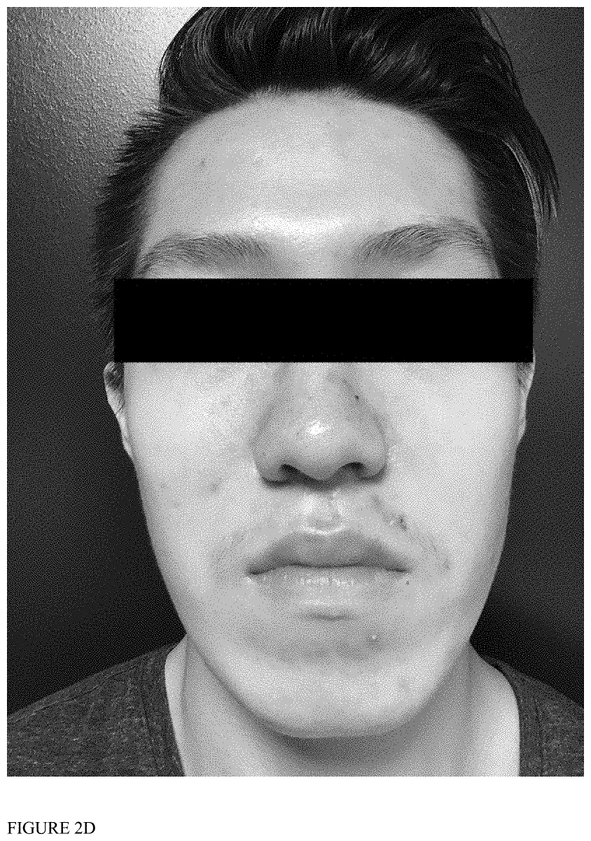

FIGS. 1A-1B depict before and after photos of Patient 1**, in the fifth chart above, being administered a cream comprising tretinoin (0.05%), niacinamide 2%, azelaic acid 8%, and sodium hyaluronate 0.25% for approximately eight weeks. FIGS. 2A-2D depict before and after photos of Patient 1*, in the first chart above, being administered a cream comprising tretinoin (0.025%), niacinamide 2%, and sodium hyaluronate 0.25% for approximately eight weeks.

### Example 5—Rosacea Compositions

In this example, various rosacea compositions were prepared using the turmeric base prepared according to Example 1A, Example 1B, or Example 1C, following the manufacturing procedures (depending on whether it was a cream, lotion, solution, or ointment formulation) according to Example 2.

For the treatment results using the rosacea compositions in this example, below is the rosacea grading scores that are referenced in the charts.

| | Rosacea Grade | | | | | | |
|---|---|---|---|---|---|---|---|
| | 0 | 1 | 2 | 3 | 4 | 5 | 6 |
| Description | Clear | Minimal | Mild | Mild to | Moderate | Moderate to | Severe |
| | | | | Moderate | | Severe | |
| Inflammatory | None | Rare | Few | Distinct | Pronounced | Many | Numerous |
| Lesions | | | | | | | |
| Erythema | None to | Residual | Mild | Mild to | Moderate | Moderate to | Moderate to |
| | Residual | to Mild | | Moderate | | Severe | Severe |
| Telangiectasia | Non to Mild | Mild to | Mild to | Mild to | Mild to | Moderate | Moderate to |
| | to Moderate | Moderate | Moderate | Moderate | Moderate | | Severe |

### Example 5A—Azelaic Acid 15%, Niacinamide 2%

TABLE 31
A rosacea cream

| | | Azelaic | Ethyl | Propylene | |
|---|---|---|---|---|---|
| | | acid | alcohol | glycol | CeraVe |
| | Niacinamide | flakes | USP | USP | moisturing |
| Ingredient | USP powder | powder | liquid | solution | cream |
| Quantity | 2 | 15 | 14 | 5 | 64 |
| (gms) | | | | | |

The following chart shows the results of treating representative patients with the rosacea cream/solution/ointment of this example.

| | | | | cream/ |
|---|---|---|---|---|
| | | Score at | Score at | solution/ |
| Patient | Demographics | Baseline | Follow up | ointment |
| Patient 1 | 45-50 year old female | 2 | 1 | cream |
| Patient 2 | 30-35 year old female | 3 | 1 | cream |
| Patient 3 | 25-30 year old female | 2 | 0 | cream |

### Example 5B—Azelaic Acid 15%, Metronidazole 1%, Ivermectin 1%

TABLE 32
A rosacea cream

| | Ingredient | | | | | | |
|---|---|---|---|---|---|---|---|
| | | | Azelaic | | Ethyl | Propylene | CeraVe |
| | Metronidazole | Ivermectin | acid flakes | THC | alcohol | glycol USP | moisturing |
| | USP powder | powder | powder | powder | USP liquid | solution | cream |
| Quantity | 1 | 1 | 15 | 0.5 | 14 | 5 | 63.5 |
| (gms) | | | | | | | |

The following chart shows the results of treating representative patients with the rosacea cream/solution/ointment of this example.

| | | | | cream/ |
|---|---|---|---|---|
| | | Score at | Score at | solution/ |
| Patient | Demographics | Baseline | Follow up | ointment |
| Patient 1 | 65-70 year old Female | 4 | 2 | cream |
| Patient 2 | 35-40 year old male | 4 | 2 | cream |
| Patient 3 | 60-65 year old male | 3 | 1 | cream |
| Patient 4 | 70-75 year old female | 4 | 1 | cream |
| Patient 5 | 55-60 year old male | 3 | 2 | cream |
| Patient 6 | 30-35 year old female | 2 | 0 | cream |
| Patient 7 | 45-50 year old female | 2 | 1 | cream |
| Patient 8 | 70-75 year old female | 4 | 0 | cream |
| Patient 9 | 65-70 year old male | 2 | 1 | cream |
| Patient 10 | 55-60 year old female | 3 | 0 | cream |
| Patient 11 | 45-50 year old male | 2 | 0 | cream |

### Example 6—An Anti-Aging Base for a Topical Composition—a Cream Vehicle

TABLE 33
A cream vehicle for an anti-aging base

| | Quantity |
|---|---|
| Ingredient | (grams) |
| Alpha lipoic acid powder | 0.5 |
| Resveratrol | 0.25 |
| Vitamin E acetate USP liquid | 1 |
| Tetrahydrocurcuminoids powder | 0.5 |
| Ethoxy diglycol liquid | 5 |
| CeraVe moisturing cream | 92.85 |
| Sodium metabisulfite NF granule | 0.2 |

Manufacturing Procedure:
1. Sodium metabisulfite, resveratrol, lipoic acid, and THC were weighed into weigh boats and added to a mortar.
2. The triturate ingredients in mortar were dried to achieve a white powder mixture with uniform particle size.
3. Vitamin E acetate liquid and ethoxy diglycol were weighed into a syringe.
4. The dry ingredients in the mortar were wetted with the liquid in Step 3 to form a white slurry with the consistency of syrup or thick lotion.
5. CeraVe cream was weighed into a weigh boat or other tared vessel, and was added to the slurry in the mortar using geometric dilution.
6. The mixture in the mortar was mixed to create a white homogenous mixture, and transferred into an appropriately-sized Unguator® jar for further mixing for 2 cycles (2 minutes, twice)
7. The formulation was obtained as a white cream with a shiny surface.

### Example 7—Anti-Aging Compositions

In this example, various anti-aging compositions were prepared using the anti-aging base prepared according to Example 6, following the typical manufacturing procedure for a cream formulation according to Example 2, except for replacing the cream vehicle of Example 1A with the cream vehicle according to Example 6.

For the treatment results using the anti-aging compositions in this example, below is a chart showing the end assessment of treatment results. For the examples below, the score at baseline and the score at the end of the treatment, evaluated on a 1-4 scale, correspond with the Group I-IV description in the chart below, respectively. The end assessment would improve a classification, if skin texture was visibly improved even if wrinkles were unchanged in the modified scale.

| Group | Classification | Typical Age | Description | Skin Characteristics |
|---|---|---|---|---|
| I | Mild | 28-35 | No wrinkles | Early photoageing: mild pigment changes, no |
| | | | | keratosis, minimal wrinkle, minimal or no makeup |
| | | | | required |
| II | Moderate | 35-5 | Wrinkles in motion | Early to moderate photoageing: early brown spots |
| | | | | visible, Keratosis palpable but not visible, parallel |
| | | | | smile lines begin to appear, wears some foundation |
| III | Advanced | 50-66 | Wrinkles at rest | Advanced photoageing: discolouration, visible |
| | | | | capillaries, visible keratosis, wears heavier foundation |
| IV | Severe | 60 and above | Only wrinkles | Severe photoageing: yellow/grey skin colour, prior |
| | | | | skin malignancies, wrinkles throughout, no normal |
| | | | | skin, cannot wear makeup because it cracks and |
| | | | | cakes |

### Example 7A—Tretinoin (0.0125%, 0.025%, 0.05%), Niacinamide 2%

An anti-aging cream

| | | | | |
|---|---|---|---|---|
| | Tretinoin | Niacinamide | Anti-aging | Ethyl |
| | USP | USP | cream vehicle | alcohol USP |
| Ingredient | powder | powder | (Example 6) | liquid |

TABLE 34

| | | | | |
|---|---|---|---|---|
| Quantity (gms) | 0.0125 | 2 | 94.9 | 5 |

TABLE 35

| | | | | |
|---|---|---|---|---|
| Quantity (gms) | 0.025 | 2 | 92.975 | 5 |

TABLE 36

| | | | | |
|---|---|---|---|---|
| Quantity (gms) | 0.05 | 2 | 94.95 | 5 |

### Example 7B—Tretinoin (0.0125%, 0.025%, 0.05%), Niacinamide 2%, Sodium Hyaluronate 0.25%

TABLE 37
An anti-aging cream

| | Ingredient | | | | | |
|---|---|---|---|---|---|---|
| | Tretinoin | | Anti-aging | Ethyl | Sodium | |
| | USP | Niacinamide | cream vehicle | alcohol | Hyaluronate | Sterile water |
| | powder | USP powder | (Example 6) | USP liquid | powder | USP liquid |
| Quantity | 0.0125 | 2 | 80.725 | 6 | 0.25 | 11 |
| (gms) | | | | | | |

TABLE 38
An anti-aging cream

| | Ingredient | | | | | |
|---|---|---|---|---|---|---|
| | Tretinoin | | Anti-aging | Ethyl | Sodium | |
| | USP | Niacinamide | cream vehicle | alcohol | Hyaluronate | Sterile water |
| | powder | USP powder | (Example 6) | USP liquid | powder | USP liquid |
| Quantity | 0.025 | 2 | 80.725 | 6 | 0.25 | 11 |
| (gms) | | | | | | |

TABLE 39
An anti-aging cream

| | Ingredient | | | | | |
|---|---|---|---|---|---|---|
| | Tretinoin | | Anti-aging | Ethyl | Sodium | |
| | USP | Niacinamide | cream vehicle | alcohol | Hyaluronate | Sterile water |
| | powder | USP powder | (Example 6) | USP liquid | powder | USP liquid |
| Quantity | 0.05 | 2 | 79.7 | 7 | 0.25 | 11 |
| (gms) | | | | | | |

The following chart shows the results of treating representative patients with the anti-aging cream/solution/ointment of this example.

| | | | | |
|---|---|---|---|---|
| | With or without | | | Score at |
| | Hyaluronic | | Score at | End of |
| Patient | Acid | Demographics | Baseline | Treatment |

Tretinoin 0.0125%, Niacinamide 2%, Cream

| | | | | |
|---|---|---|---|---|
| Patient 1 | With HA | 45-50 year old | 3 | 2 |
| | | female | | |
| Patient 2 | With HA | 50-55 year old | 3 | 2 |
| | | female | | |
| Patient 3 | With HA | 40-45 year old | 2 | 1 |
| | | female | | |
| Patient 4 | With HA | 45-50 year old | 2 | 1 |
| | | female | | |

Tretinoin 0.025%, Niacinamide 2%, Cream

| | | | | |
|---|---|---|---|---|
| Patient 1 | with HA | 50-55 year old | 3 | 2 |
| | | female | | |
| Patient 2 | with HA | 55-60 year old | 3 | 2 |
| | | female | | |
| Patient 3 | with HA | 35-40 year old | 2 | 1 |
| | | female | | |
| Patient 4 | with HA | 50-55 year old | 3 | 2 |
| | | female | | |
| Patient 5 | with HA | 55-60 year old | 3 | 2 |
| | | female | | |
| Patient 6 | with HA | 60-65 year old | 3 | 2 |
| | | female | | |
| Patient 7 | with HA | 60-65 year old | 3 | 2 |
| | | female | | |
| Patient 8 | with HA | 55-60 year old | 2 | 1 |
| | | female | | |
| Patient 9 | with HA | 45-50 year old | 2 | 1 |
| | | female | | |
| Patient 10 | with HA | 40-45 year old | 2 | 1 |
| | | female | | |

Tretinoin 0.05%, Niacinamide 2%, Cream

| | | | | |
|---|---|---|---|---|
| Patient 1 | with HA | 40-45 year old | 2 | 1 |
| | | female | | |
| Patient 2 | with HA | 45-50 year old | 2 | 1 |
| | | female | | |
| Patient 3 | with HA | 40-45 year old | 2 | 1 |
| | | female | | |
| Patient 4 | with HA | 50-55 year old | 3 | 2 |
| | | female | | |
| Patient 5 | with HA | 55-60 year old | 3 | 2 |
| | | female | | |
| Patient 6 | with HA | 50-55 year old | 2 | 1 |
| | | female | | |
| Patient 7 | with HA | 55-60 year old | 2 | 1 |
| | | female | | |
| Patient 8 | with HA | 45-50 year old | 2 | 1 |
| | | female | | |
| Patient 9 | with HA | 40-45 year old | 2 | 1 |
| | | female | | |
| Patient 10 | with HA | 35-40 year old | 2 | 1 |
| | | female | | |

### Example 8—Composition for Treating Warts

TABLE 40
A wart solution

| | Quantity |
|---|---|
| Ingredient | (grams) |
| 5-fluorouracil USP powder | 5 |
| Salicylic acid solution (50%) | 60 |
| Triethanolamine NF liquid | 35 |
| Sodium metabisulfite NF granule | 0.2 |

Manufacturing Procedure:
1. Sodium metabisulfite and 5-fluorouracil were weighed into weigh boats.
2. Trolamine (triethanolamine) and 50% salicylic acid solution were weighed in syringes or beaker, and both liquids were combined in an appropriately sized beaker.
3. Both 5-fluorouracil and sodium metabisulfite as well as an appropriate stir bar were added to the beaker.
4. The beaker was placed on the CIMAREK device, which was placed under the hood to avoid toxic vapors, and was stirred without heating.
5. When the solution was completely clear, visual particulate test was performed using black/white background to assure complete dissolution of solids.
6. The formulation was obtained as a clear viscous liquid, and was dispensed into brown glass applicator bottle.

The following chart shows the results of treating representative patients with the wart solution of this example.

5-Fluorouracil 5%, Salicylic Acid 30%, Solution

| | | Score at | Score at End |
|---|---|---|---|
| Patient | Demographics | Baseline * | of Treatment * |
| Patient 1 | 10-15 year old | 2 | 1 |
| | male | | |
| Patient 2 | 15-20 year old | 2 | 0 |
| | female | | |
| Patient 3 | 20-25 year old | 2 | 1 |
| | female | | |
| Patient 4 | 10-15 year old | 1 | 0 |
| | female | | |
| Patient 5 | 35-40 year old | 2 | 1 |
| | female | | |
| Patient 6 | 25-30 year old | 2 | 1 |
| | female | | |

*Assessment score:
2-wart is thickened and palpable;
1-wart is only perceptible by black dots and not thickened or palpable;
0-clear skin or ulcer where wart was present indicating wart is destroyed and skin is healing.

### Example 9—Composition for Treating Warts

TABLE 41
A wart paste

| Ingredient | Quantity |
|---|---|
| | (grams) |
| 5-fluorouracil USP powder | 5 |
| Salicylic acid USP powder | 70 |
| Triethanolamine NF liquid | 8 |
| Polysorbate 20 NF liquid | 8 |
| Sterile water USP liquid | 2.5 |
| Dimethyl sulfoxide USP liquid | 2.5 |
| Lanolin USP (anhydrous) | 7 |

Manufacturing Procedure:
1. Salicylic acid, 5-fluorouracil, and lanolin were weighed into weigh boats.
2. Salicylic acid and 5-fluorouracil were placed into an appropriately sized mortar, and the triturate were dried to reduce particle size, resulting in a white, homogenous powder mixture.
3. Trolamine, polysorbate 20, water, and DMSO were weighed in syringes or appropriate vessels and were combined into a single vessel and mixed.
4. The powers in the mortar were wetted with the liquids to form a homogenous product.
5. When the mixture was homogenous, lanolin was added and mixed in the mortar.
6. The resulting mixture was milled multiple times until achieving a smooth, homogenous, grain-free white paste.
7. The formulation was obtained as a thick white paste, and was dispensed into an airless pump dispenser.

The following chart shows the results of treating representative patients with the wart paste of this example.

5-Fluorouracil 5%, Salicylic Acid 70%, Paste

| | | Score at | Score at End |
|---|---|---|---|
| Patient | Demographics | Baseline * | of Treatment * |
| Patient 1† | 10-15 year old male | 2 | 0 |
| Patient 2‡ | 30-35 year old male | 2 | 0 |
| Patient 3 | 15-20 year old | 2 | 1 |
| | female | | |
| Patient 4 | 10-15 year old | 2 | 0 |
| | female | | |
| Patient 5 | 5-10 year old male | 2 | 1 |
| Patient 6 | 25-30 year old male | 2 | 1 |
| Patient 7 | 40-45 year old | 2 | 0 |
| | female | | |
| Patient 8 | 20-25 year old male | 2 | 1 |
| Patient 9 | 10-15 year old | 2 | 0 |
| | female | | |
| Patient 10 | 10-15 year old male | 2 | 0 |

*Assessment score:
2-wart is thickened and palpable;
1-wart is only perceptible by black dots and not thickened or palpable;
0-clear skin or ulcer where wart was present indicating wart is destroyed and skin is healing.

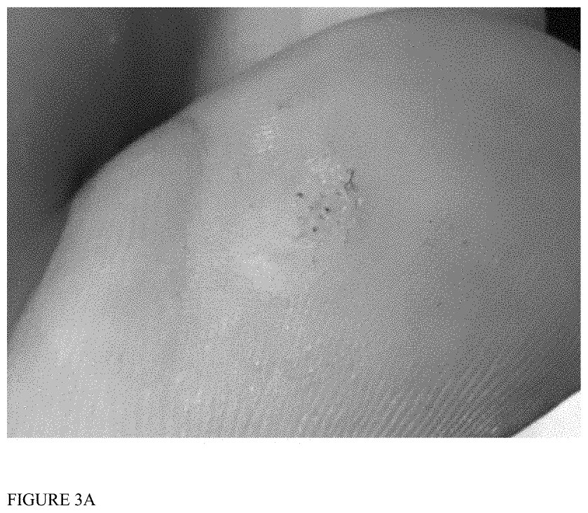

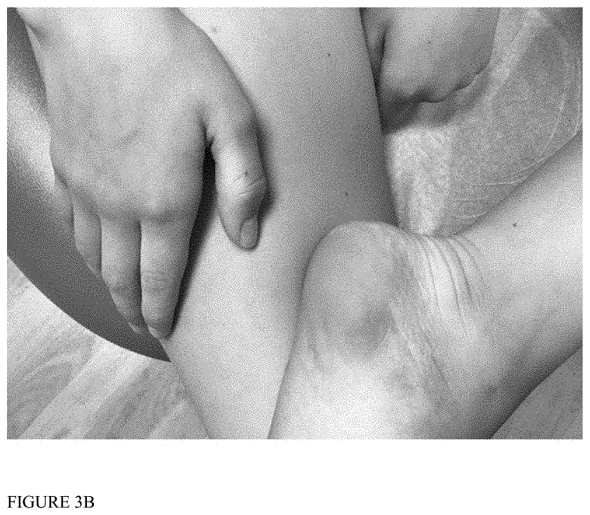

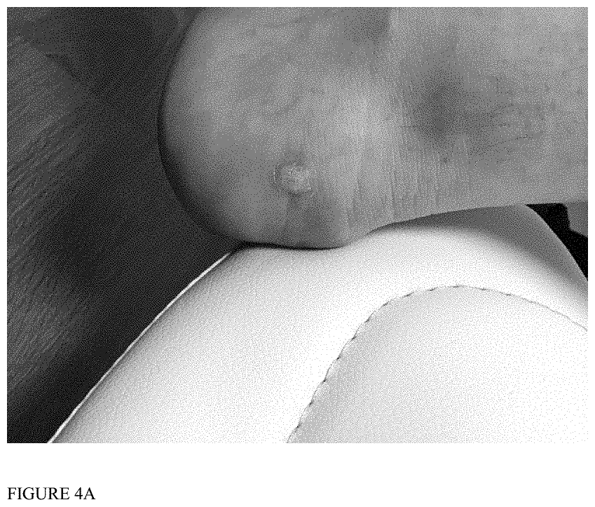

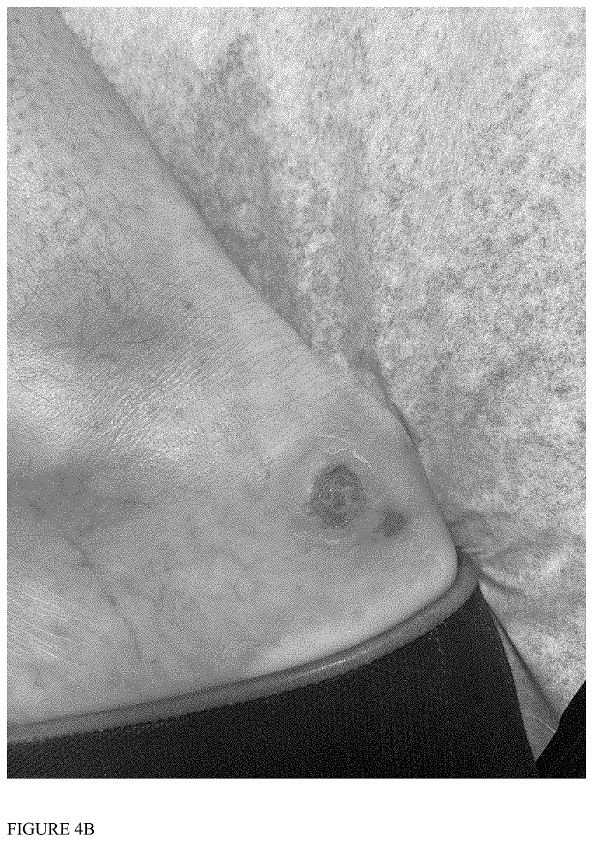

FIGS. 3A-3B depict before and after photos of Patient 1†, in the chart above, being administered a wart paste comprising 5-fluorouracil 5%, and salicylic Acid 70% for approximately four weeks. FIGS. 4A-4B depict before and after photos of Patient 2‡, in the chart above, being administered a wart paste comprising 5-fluorouracil 5%, and salicylic Acid 70% for approximately four weeks.

1. A topical composition, comprising:
a suitable base carrying an antioxidant component comprising turmeric or its derivative.

2. The topical composition of claim 1, wherein the turmeric or its derivative is tetrahydrocurcuminoids.

3. The topical composition of claim 2, wherein the topical composition is a cream formulation comprising a cream base containing:
about 0.1-1 wt % tetrahydrocurcuminoids,
about 1-6 wt % ethoxy diglycol or propylene glycol,
about 0-9 wt % ethyl alcohol,
about 0-0.5 wt % sodium metabisulfite, and
about 83.5-98.9 wt % a moisturizing cream,
wherein the amount is based on the total amount of the cream base.

4. The topical composition of claim 2, wherein the topical composition is a lotion formulation comprising a lotion base containing:
about 0.1-1 wt % tetrahydrocurcuminoids,
about 5-9 wt % ethyl alcohol,
about 0-0.5 wt % sodium metabisulfite, and
about 89.5-94.9 wt % a moisturizing lotion,
wherein the amount is based on the total amount of the lotion base.

5. The topical composition of claim 2, wherein the topical composition is an ointment formulation comprising an ointment base containing:
about 0.1-1 wt % tetrahydrocurcuminoids,
about 1-5 wt % ethoxy diglycol or propylene glycol,
about 87-98.9 wt % petrolatum, and
about 0-7 wt % mineral oil,
wherein the amount is based on the total amount of the ointment base.

6. The topical composition of claim 2, wherein the topical composition is a solution formulation comprising a solution base containing:
about 0.1-1 wt % tetrahydrocurcuminoids,
about 1-5 wt % dimethyl sulfoxide,
about 49-50 wt % ethyl alcohol, and
about 45-48.9 wt % propanediol,
wherein the amount is based on the total amount of the solution base.

7. The topical composition of claim 3, wherein the topical composition is an anti-inflammatory composition, further comprising:
a) clobetasol and niacinamide,
b) fluocinodide and niacinamde,
c) triamcinolone acetonide and niacinamde, or
d) desonide and niacinamde.

8. The topical composition of claim 7, wherein the amount of niacinamide is about 1.5-2.5 wt % of the total amount of the topical composition.

9. The topical composition of claim 7, wherein the amount of clobetasol, fluocinodide, triamcinolone acetonide, or desonide, each when present, is about 0.01-0.2 wt % of the total amount of the topical composition.

10. The topical composition of claim 7, wherein the anti-inflammatory composition comprises
a) about 0.05 or 0.1 wt % clobetasol propionate and about 2 wt % niacinamide,
b) about 0.05 wt % fluocinodide and about 2 wt % niacinamde,
c) about 0.1 wt % triamcinolone acetonide and about 2 wt % niacinamde, or
d) about 0.05 wt % desonide and about 2 wt % niacinamde,
wherein the amount is based on the total amount of the topical composition.

11. The topical composition of claim 3, wherein the topical composition is an acne composition, further comprising:
a) tretinoin and niacinamide, or
b) tretinoin, niacinamide, and acelaic acid, and
optionally, hyaluronic acid or a salt thereof and water.

12. The topical composition of claim 11, wherein the amount of niacinamide is about 1.5-2.5 wt % of the total amount of the topical composition.

13. The topical composition of claim 11, wherein the amount of tretinoin is about 0.01-0.2 wt % of the total amount of the topical composition.

14. The topical composition of claim 11, wherein the amount of acelaic acid, when present, is about 5-10 wt % of the total amount of the topical composition.

15. The topical composition of claim 11, wherein the amount of hyaluronic acid or a salt thereof, when present, is about 0.2-0.3 wt % of the total amount of the topical composition.

16. The topical composition of claim 11, wherein the acne composition comprises
a) about 0.025, 0.05, or 0.1 wt % tretinoin and about 2 wt % niacinamide, or
b) about 0.025, 0.05, or 0.1 wt % tretinoin, about 2 wt % niacinamide, and about 8 wt % acelaic acid, and
optionally, about 0.25 wt % sodium hyaluronate,
wherein the amount is based on the total amount of the topical composition.

17. The topical composition of claim 3, wherein the topical composition is a rosacea composition, further comprising:
a) acelaic acid and niacinamide, or
b) acelaic acid, metronidazole, and ivermectin.

18. The topical composition of claim 17, wherein the amount of acelaic acid is about 10-20 wt % of the total amount of the topical composition.

19. The topical composition of claim 17, wherein the amount of niacinamide, when present, is about 1.5-2.5 wt % of the total amount of the topical composition.

20. The topical composition of claim 17, wherein the amount of metronidazole or ivermectin, each when present, is about 0.5-1.5 wt % of the total amount of the topical composition.

21. The topical composition of claim 17, wherein the rosacea composition comprises
a) about 15 wt % acelaic acid and about 2 wt % niacinamide, or
b) about 15 wt % acelaic acid, about 1 wt % metronidazole, and about 1 wt % ivermectin,
wherein the amount is based on the total amount of the topical composition.

22-44. (canceled)
